(19) United States
(12) Patent Application Publication
(10) Pub. No.: US 2023/0065640 A1
(43) Pub. Date: Mar. 2, 2023

(54) COMBINATION THERAPIES AND BIOMARKERS FOR TREATING B-CELL LYMPHOMAS

(71) Applicants: Xiang LI, Beijing (CN); Yiyou CHEN, San Jose, CA (US)

(72) Inventors: Xiang LI, Beijing (CN); Yiyou CHEN, San Jose, CA (US)

(21) Appl. No.: 17/797,621

(22) PCT Filed: Feb. 5, 2021
(86) PCT No.: PCT/US2021/016863

§ 371 Date: Aug. 4, 2022

(30) Foreign Application Priority Data
Feb. 7, 2020 (CN) PCT/CN2020/074516

(51) Int. Cl. A61K 31/497; C12Q 1/6886; C07K 16/28; A61K 31/4184; A61K 31/502; A61P 35/00

(52) U.S. Cl. CPC A61K 31/497; C12Q 1/6886; C07K 16/2887; A61K 31/4184; A61K 31/502; A61P 35/00

(57) ABSTRACT

Provided are combination therapies and secondary biomarkers for treating MYCC-associated diffuse large B-cell lymphoma (DLBCL) with YM155-based therapies, and related kits, compositions, and methods of use thereof.

### CROSS-REFERENCE TO RELATED APPLICATIONS

This application claims priority to PCT/CN2020/074516, filed Feb. 7, 2020, which is incorporated by reference in its entirety.

### BACKGROUND

### Technical Field

Embodiments of the present disclosure relates to combination therapies and secondary biomarkers for treating MYCC-associated diffuse large B-cell lymphoma (DLBCL) with YM155-based therapies, and related kits, compositions, and methods of use thereof.

### Description of the Related Art

YM155 monobromide is a small-molecule survivin inhibitor that induces the down-regulation of survivin and exhibits potent antitumor activity (see, e.g., Minematsu et al., Drug Metabolism and Disposition, 37:619-628, 2008). YM-155 exerts anti-tumor effects in various in vivo cancer models, including prostate, pancreatic, and lung cancer (see, e.g., Nakahara et al., Cancer Research 67:8014-8021, 2007; and Na et al., PLoS One 7(6), 2012).

Diffuse large B-cell lymphoma (DLBCL) is a common type of aggressive non-Hodgkin lymphoma (NHL). The disease is heterogeneous clinically, morphologically, and molecularly. Insights into the molecular heterogeneity of DLBCL are beginning to yield therapeutics with significant promise for key subsets of patients

However, there is a need in the art to identify improved biomarkers and combination therapies for treating DLBCL.

### BRIEF SUMMARY

Embodiments of the present disclosure include methods for treating a MYCC-associated diffuse large B-cell lymphoma (DLBCL) in a subject in need thereof, comprising

administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, thereby treating the MYCC-associated DLBCL in the subject in need thereof.

Certain embodiments comprise

(a) determining MYCC gene copy number, or MYCC gene chromosomal location site, in a DLBCL sample from the subject; and

(b) administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, to the subject if MYCC gene copy number in the DLBCL sample is increased relative to that of a MYCC gene copy number reference, or if MYCC gene chromosomal location site in the DLBCL sample is translocated relative to that of a MYCC gene chromosomal location site reference.

Certain embodiments comprise administering to the subject a chemotherapeutic agent excluding (or other than) YM155 monobromide if MYCC gene copy number in the DLBCL sample is not substantially increased relative to that of the MYCC gene copy number reference, or if MYCC gene chromosomal location site in the DLBCL sample is not translocated relative to that of the MYCC gene chromosomal location site reference. Certain embodiments comprise administering YM155, or an analog or derivative thereof, to the subject in combination with a second anti-cancer agent selected from CHOP (cyclophosphamide, doxorubicin, vincristine, and prednisone), an anti-CD20 antibody, bendamustine, and an PARP inhibitor, including combinations thereof, optionally R-CHOP (ritixumab+CHOP).

In some embodiments, the anti-CD20 antibody is selected from one or more of rituximab, ofatumumab, ocrelizumab, Iodine-131 tositumomab, obinutuzumab, and ibritumomab, or wherein the PARP inhibitor is selected from one or more of olaparib, rucaparib, nirparib, talazoparib, talazoparib, veliparib, and pamiparib. In some embodiments, the MYCC gene copy number in the DLBCL sample is increased by about or at least about 1.5, 2, 3, 4, 5, 6, 7, 8, 9, or 10-fold relative to that of the MYCC gene copy number reference.

Certain embodiments comprise determining MYCC gene copy number in the DLBCL sample by array comparative genome hybridization (aCGH), single nucleotide polymorphism (SNP) array, copy number variation (CNV) sequencing, or multiplex ligation-dependent probe amplification (MLPA). Certain embodiments comprise determining MYCC gene chromosomal location site in the DLBCL sample by in situ hybridization (ISH), fluorescence in situ hybridization (FISH), next generation sequencing (NGS), or comparative genome hybridization (CGH). Certain embodiments comprise obtaining the MYCC gene copy number reference from a database, or determining the MYCC gene copy number reference from a non-cancerous tissue (optionally B-cell) from a control, optionally by aCGH, SNP array, CNV sequence, or MLPA. Certain embodiments comprise obtaining the MYCC gene chromosomal location site reference from a database, or determining the MYCC gene chromosomal location site reference from a non-cancerous tissue (optionally B-cell) from a control, optionally by ISH, FISH, NGS, or CGH.

Certain embodiments comprise obtaining the DLBCL sample from the subject. In some embodiments, the DLBCL sample is a surgical sample, a biopsy sample, a pleural effusion sample, or an ascetic fluid sample obtained from the subject, optionally selected from one or more of blood, lymph node, and bone marrow. In some embodiments, the subject is a human subject. In some embodiments, the DLBCL is selected from DLBCL not otherwise specified (DLBCL-NOS), T-cell/histiocyte-rich B-cell lymphoma, primary or secondary DLBCL of the central nervous system (CNS), primary cutaneous DLBCL (leg type), and Epstein-Barr virus (EBV)-positive DLBCL optionally of the elderly. In some embodiments, the DLBCL is refractory to prior therapy, optionally selected from anthracycline-based therapy, CHOP, anti-CD20 therapy (optionally rituximab), R-CHOP (ritixumab+CHOP), radiation therapy, etoposide (optionally R-EPOCH), and autologous stem cell transplant (ASCT), including combinations thereof. In some embodiments, the DLBCL expresses or overexpresses BCL2, BCL6, or both, and/or wherein the DLBCL comprises an alteration of the BCL2 gene (18q21 locus), an alteration of the BCL-6 gene (3q27 locus), or both.

Certain embodiments further comprise

(c) determining BCL2 and/or BCL6 expression in the DLBCL sample from the subject, and/or determining alterations of the BCL2 gene (18q21 locus) and/or the BCL6 gene (3q27 locus) in the DLBCL sample from the subject. Certain embodiments further comprise administering a second anti-cancer agent comprising a BCL2 inhibitor to the subject if the DLBCL expresses or overexpresses BCL2, and/or if the DLBCL comprises an alteration of the BCL2 gene (18q21 locus), optionally wherein the BCL2 inhibitor is selected from ABT199 (venetoclax), ABT-263 (navitoclax), ABT-737, GX-15-070 (obatoclax), GDC-0199, BP1002 (antisense), AT-101, and SPC2996.

In some embodiments, the YM155 monobromide, or the analog or derivative thereof, and the second anti-cancer agent are administered separately or sequentially. In some embodiments, the YM155 monobromide, or the analog or derivative thereof, and the second anti-cancer agent are administered together at the same time.

Also included are methods for treating a MYCC/BCL2-associated diffuse large B-cell lymphoma (DLBCL) in a subject in need thereof, comprising

administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, thereby treating the MYCC/BCL2-associated DLBCL in the subject in need thereof.

Certain embodiments comprise

(a) determining MYCC gene copy number, or MYCC gene chromosomal location site, in the DLBCL sample from the subject;

(b) determining BCL2 expression in the DLBCL sample from the subject, and/or determining alterations of the BCL2 gene (18q21 locus) in the DLBCL sample from the subject; and

(c) administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, to the subject if MYCC gene copy number in the DLBCL sample is increased relative to that of a MYCC gene copy number reference, or if MYCC gene chromosomal location site in the DLBCL sample is translocated relative to that of a MYCC gene chromosomal location site reference, and if the DLBCL expresses or overexpresses BCL2, and/or if the DLBCL comprises an alteration of the BCL2 gene (18q21 locus).

Certain embodiments comprise administering YM155, or an analog or derivative thereof, to the subject in combination with a second anti-cancer agent selected from a BCL2 inhibitor, CHOP (cyclophosphamide, doxorubicin, vincristine, and prednisone), an anti-CD20 antibody, bendamustine, and an PARP inhibitor, including combinations thereof, optionally R-CHOP (ritixumab+CHOP).

In some embodiments, the BCL2 inhibitor is selected from ABT199 (venetoclax), ABT-263 (navitoclax), ABT-737, GX-15-070 (obatoclax), GDC-0199, BP1002 (antisense), AT-101, and SPC2996. In some embodiments, the anti-CD20 antibody is selected from one or more of rituximab, ofatumumab, ocrelizumab, Iodine-131 tositumomab, obinutuzumab, and ibritumomab, or wherein the PARP inhibitor is selected from one or more of olaparib, rucaparib, nirparib, talazoparib, talazoparib, veliparib, and pamiparib. In some embodiments, the MYCC gene copy number in the DLBCL sample is increased by about or at least about 1.5, 2, 3, 4, 5, 6, 7, 8, 9, or 10-fold relative to that of the MYCC gene copy number reference.

Certain embodiments comprise determining MYCC gene copy number in the DLBCL sample by array comparative genome hybridization (aCGH), single nucleotide polymorphism (SNP) array, copy number variation (CNV) sequencing, or multiplex ligation-dependent probe amplification (MLPA). Certain embodiments comprise determining MYCC gene chromosomal location site in the DLBCL sample by in situ hybridization (ISH), fluorescence in situ hybridization (FISH), next generation sequencing (NGS), or comparative genome hybridization (CGH). Certain embodiments comprise obtaining the MYCC gene copy number reference from a database, or determining the MYCC gene copy number reference from a non-cancerous tissue (optionally B-cell) from a control, optionally by aCGH, SNP array, CNV sequence, or MLPA. Certain embodiments comprise obtaining the MYCC gene chromosomal location site reference from a database, or determining the MYCC gene chromosomal location site reference from a non-cancerous tissue (optionally B-cell) from a control, optionally by ISH, FISH, NGS, or CGH. Certain embodiments comprise obtaining the DLBCL sample from the subject.

In some embodiments, the DLBCL sample is a surgical sample, a biopsy sample, a pleural effusion sample, or an ascetic fluid sample obtained from the subject, optionally selected from one or more of blood, lymph node, and bone marrow. In some embodiments, the subject is a human subject. In some embodiments, the DLBCL is selected from DLBCL not otherwise specified (DLBCL-NOS), T-cell/histiocyte-rich B-cell lymphoma, primary or secondary DLBCL of the central nervous system (CNS), primary cutaneous DLBCL (leg type), and Epstein-Barr virus (EBV)-positive DLBCL optionally of the elderly. In some embodiments, the DLBCL is refractory to prior therapy, optionally selected from anthracycline-based therapy, CHOP, anti-CD20 therapy (optionally rituximab), R-CHOP (ritixumab+CHOP), radiation therapy, etoposide (optionally R-EPOCH), and autologous stem cell transplant (ASCT), including combinations thereof.

Also included are patient care kits, comprising

(a) means for measuring MYCC gene copy number, or MYCC gene chromosomal location site, in a diffuse large B-cell lymphoma (DLBCL) sample of tissue from a subject, including cancer tissue and non-cancerous tissue;

(b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof; and

(c) a second anti-cancer agent selected from CHOP (cyclophosphamide, doxorubicin, vincristine, and prednisone), an anti-CD20 antibody, bendamustine, a PARP inhibitor, and a BCL2 inhibitor, including combinations thereof, optionally R-CHOP (ritixumab+CHOP).

In some embodiments, the anti-CD20 antibody is selected from one or more of rituximab, ofatumumab, ocrelizumab, Iodine-131 tositumomab, obinutuzumab, and ibritumomab, or wherein the PARP inhibitor is selected from one or more of olaparib, rucaparib, nirparib, talazoparib, talazoparib, veliparib, and pamiparib, or wherein the BCL2 inhibitor is selected from ABT199 (venetoclax), ABT-263 (navitoclax), ABT-737, GX-15-070 (obatoclax), GDC-0199, BP1002 (antisense), AT-101, and SPC2996. In some embodiments, the means for measuring MYCC gene copy number comprise reagents for performing a diagnostic assay selected from one or more of array comparative genome hybridization (aCGH), single nucleotide polymorphism (SNP) array, copy number variation (CNV) sequencing, and multiplex ligation-dependent probe amplification (MLPA) on a human MYCC gene.

In some embodiments, the means for measuring MYCC gene chromosomal location site comprise reagents for performing a diagnostic assay selected from one or more of in situ hybridization (ISH), fluorescence in situ hybridization (FISH), next generation sequencing (NGS), and comparative genome hybridization (CGH) on a human MYCC gene. Certain kits comprise a MYCC gene copy number reference value obtained from a database, or determined from a non-cancerous tissue from a control. Certain kits comprise a MYCC gene chromosomal location site reference obtained from a database, or determined from a non-cancerous tissue from a control. Certain kits further comprise (d) means for determining BCL2 expression in the sample of tissue from the subject, and/or means for determining alterations of the BCL2 gene (18q21 locus) in the sample of tissue from the subject.

Also included are patient care kits, comprising

(a) means for measuring MYCC gene copy number, or MYCC gene chromosomal location site, in a diffuse large B-cell lymphoma (DLBCL) sample of tissue from a subject, including cancer tissue and non-cancerous tissue;

(b) means for determining BCL2 expression in the DLBCL sample of tissue from the subject, and/or means for determining alterations of the BCL2 gene (18q21 locus) in the sample of tissue from the subject; and

(c) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof.

Certain kits comprise (d) a second anti-cancer agent selected from CHOP (cyclophosphamide, doxorubicin, vincristine, and prednisone), an anti-CD20 antibody, bendamustine, a PARP inhibitor, and a BCL2 inhibitor, including combinations thereof, optionally R-CHOP (ritixumab+CHOP).

In some embodiments, the anti-CD20 antibody is selected from one or more of rituximab, ofatumumab, ocrelizumab, Iodine-131 tositumomab, obinutuzumab, and ibritumomab, or wherein the PARP inhibitor is selected from one or more of olaparib, rucaparib, nirparib, talazoparib, talazoparib, veliparib, and pamiparib, or wherein the BCL2 inhibitor is selected from ABT199 (venetoclax), ABT-263 (navitoclax), ABT-737, GX-15-070 (obatoclax), GDC-0199, BP1002 (antisense), AT-101, and SPC2996.

In some embodiments, the means for measuring MYCC gene copy number comprise reagents for performing a diagnostic assay selected from one or more of array comparative genome hybridization (aCGH), single nucleotide polymorphism (SNP) array, copy number variation (CNV) sequencing, and multiplex ligation-dependent probe amplification (MLPA) on a human MYCC gene. In some embodiments, the means for measuring MYCC gene chromosomal location site comprise reagents for performing a diagnostic assay selected from one or more of in situ hybridization (ISH), fluorescence in situ hybridization (FISH), next generation sequencing (NGS), and comparative genome hybridization (CGH) on a human MYCC gene

Certain kits comprise a MYCC gene copy number reference value obtained from a database, or determined from a non-cancerous tissue from a control. Certain kits comprise a MYCC gene chromosomal location site reference obtained from a database, or determined from a non-cancerous tissue from a control.

### BRIEF DESCRIPTION OF THE DRAWINGS

FIG. 1 shows the chemical structure of YM155 monobromide (CAS 781661-94-7).

FIG. 2 and FIG. 3 provide genomic information for the human MYC gene (see uswest.ensembl.org/Homo_sapiens/Location/View?db=core;g=ENSG00000136997;r=8:127735434-127742951).

FIG. 4 shows that DLBCL with a MYCC rearrangement is more sensitive to YM155 (PC-002) than DLBCL without the rearrangement.

FIG. 5A shows that YM155 (PC-002) induces apoptosis of DLBCL cells with a MYCC rearrangement. FIG. 5B shows that YM155 (PC-002) lowers the MYCC expression in DLBCL cells with a MYCC rearrangement.

FIG. 6 shows that YM155 (PC-002) causes tumor regression in a cell-line derived xenograft (CDX) mouse model of DLBCL with a MYCC rearrangement, alone and in combination with a PARP inhibitor (olaparib). The PARP inhibitor alone had no significant effect on tumor growth.

FIG. 7 shows YM155 (PC-002) causes tumor regression in a CDX mouse model of DLBCL with a MYCC rearrangement, alone and in combination with CHOP, the standard of care for DLBCL. CHOP therapy alone had only a moderate effect on tumor growth.

FIG. 8 shows that YM155 (PC-002) causes complete tumor regression in a CDX mouse model of DLBCL with a MYCC rearrangement and BCL2 expression (MYCC/BCL2 associated lymphoma), alone and in combination with a BCL2 inhibitor (ABT199). The BCL2 inhibitor alone had no significant effect on tumor growth.

FIG. 9 shows the results of YM155 (PC-002) treatment in a CDX model of DLBCL with a MYCC rearrangement (b/c-nu), alone and in combination with CHOP. Group 01: Vehicle. Group 02: PC-002, 5 mg/kg, QD*5*3w, D1-5, i.p., w1-3; 5 mg/kg, QD*5*3w, D1-5, i.p. w4-7. Group 03: CHOP, cyclophosphamide, 40 mg/kg, QW*3, D1, i.v., doxorubicin, 3.3 mg/kg, QW*3, D1, i.v., vincristine, 0.5 mg/kg, QW*3, D1, i.v., prednisone, 0.2 mg/kg, QD*5*3W,D1-5, p.o.; PC-002, 5 mg/kg, QD*5*3w, D1-5, i.p. w4-7. Group 04: CHOP+PC-002, cyclophosphamide, 40 mg/kg, QW*3, D1, i.v., doxorubicin, 3.3 mg/kg, QW*3, D1, i.v., vincristine, 0.5 mg/kg, QW*3, D1, i.v., prednisone, 0.2 mg/kg, QD*5*3W,D1-5, p.o., PC-002, 5 mg/kg, QD*5*3w, D1-5, i.p.

FIG. 10 shows the results of YM155 (PC-002) treatment in a SU-DHL-4 (double hit (MYCC/BCL2), CD20 positive) CDX model (b/c-nu), alone and in combination with CHOP or RCHOP (rituximab+CHOP). CHOP: cyclophosphamide, 40 mg/kg, QW*3, D1, i.v., doxorubicin, 3.3 mg/kg, QW*3, D1, i.v., vincristine, 0.5 mg/kg, QW*3, D1, i.v., prednisone, 0.2 mg/kg, QD*5*3W,D1-5, p.o. RCHOP: rituximab, 25 mg/kg, QW*3, i.p., cyclophosphamide, 40 mg/kg, QW*3, D1, i.v., doxorubicin, 3.3 mg/kg, QW*3, D1, i.v., vincristine, 0.5 mg/kg, QW*3, D1, i.v., prednisone, 0.2 mg/kg, QD*5*3W,D1-5, p.o. PC-002: 5 mg/kg, QD*5*3w, D1-5, i.p.

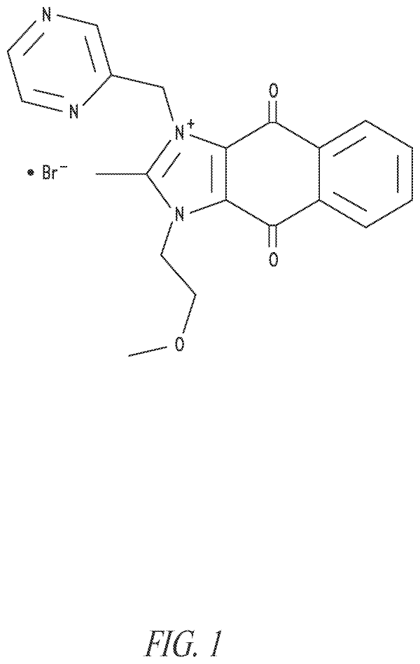

### DETAILED DESCRIPTION

Embodiments of the present disclosure are based in part on the unexpected discovery that YM155-based therapies in combination with certain other anti-cancer agents, such as CHOP (cyclophosphamide, doxorubicin, vincristine, and prednisone), rituximab, bendamustine, and/or PARP inhibitors, result in significantly increased cancer cell-killing activity in MYCC-associated, diffuse large B-cell lymphoma (DLBCL) models. Also included is the unexpected discovery that BCL2 and/or BCL6 biomarkers can be used in combination with MYCC rearrangement biomarkers (e.g., amplifications, translocations) to identify DLBCL patients that will respond optimally to YM155 therapy, including combination therapy with YM155 and other anti-cancer agents such as BLC2 inhibitors. Thus, the YM155-based combination therapies and biomarkers described herein could provide significant utility in the treatment of MYCC-associated DLBCL.

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

For the purposes of the present disclosure, the following terms are defined below.

The articles “a” and “an” are used herein to refer to one or to more than one (i.e., to at least one) of the grammatical object of the article. By way of example, “an element” means one element or more than one element.

An “antagonist” or “inhibitor” refers to biological structure or chemical agent that interferes with or otherwise reduces the physiological action of another molecule, such as a protein (e.g., survivin). In some instances, the antagonist or inhibitor specifically binds to the other molecule and/or a functional ligand of the other molecule. In some instances, the antagonist or inhibitor down-regulates the expression of the other molecule (e.g., survivin). Included are full and partial antagonists.

An “agonist” or “activator” refers to biological structure or chemical agent that increases or enhances the physiological action of another agent or molecule. In some instances, the agonist specifically binds to the other agent or molecule. Included are full and partial agonists.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The “half maximal inhibitory concentration” (or “IC₅₀”) is a measure of the potency of an agent in inhibiting a specific biological or biochemical function. This quantitative measure indicates how much of a particular agent (inhibitor) is needed to inhibit a given biological process (or component of a process, i.e. an enzyme, cell, cell receptor or microorganism) by half. The values are typically expressed as molar concentration. The concentration is commonly used as a measure of antagonist drug potency in pharmacological research. In some instances, IC₅₀ represents the concentration of an agent that is required for 50% inhibition in vitro. The IC₅₀ of an agent can be determined by constructing a dose-response curve and examining the effect of different concentrations of the agent on the desired activity, for example, inhibition of tumor cell proliferation, tumor-cell killing.

An “increased” or “enhanced” amount is typically a “statistically significant” amount, and may include an increase that is about or at least about 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 50, 60, 70, 80, 90, 100, 200, 300, 400, 500, or 1000 fold, or about or at least about 5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, relative to that of a reference or control (including all integers and ranges in between). A “decreased” or “reduced” amount is typically a “statistically significant” amount, and may include a decrease that is about or at least about 1.2, 1.4, 1.6, 1.8, 2, 3, 4, 5, 6, 7, 8, 9, 10, 15, 20, 30, 50, 60, 70, 80, 90, 100, 200, 300, 400, 500, 1000 fold, or about or at least about 5%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, relative to that of a reference or control (including all integers and ranges in between).

The term “polynucleotide” and “nucleic acid” includes mRNA, RNA, cRNA, cDNA, and DNA including genomic DNA. The term typically refers to polymeric form of nucleotides of at least 10 bases in length, either ribonucleotides or deoxynucleotides or a modified form of either type of nucleotide. The term includes single and double stranded forms of DNA.

A “gene” refers to a hereditary unit consisting of a sequence of DNA that occupies a specific location on a chromosome and codes for a functional molecule or protein. The structure of a gene consists of many elements of which the actual protein coding sequence is often only a small part. These elements include DNA regions that are not transcribed as well as untranslated regions of the RNA. Additionally, genes can have expression-altering regulatory regions that lie many kilobases upstream or downstream of the coding sequence. The information in a gene can also be represented by (or found in) a sequence of RNA or encoded protein.

A “subject” or a “subject in need thereof” includes a mammalian subject such as a human subject.

By “statistically significant” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95% or greater of some given quantity.

“Therapeutic response” refers to improvement of symptoms (whether or not sustained) based on the administration of the therapeutic response.

As used herein, the terms “therapeutically effective amount”, “therapeutic dose,” “prophylactically effective amount,” or “diagnostically effective amount” is the amount of an agent needed to elicit the desired biological response following administration.

As used herein, “treatment” of a subject (e.g. a mammal, such as a human) or a cell is any type of intervention used in an attempt to alter the natural course of the subject or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

Embodiments of the present disclosure include methods for treating a MYCC-associated diffuse large B-cell lymphoma (DLBCL) in a subject in need thereof, comprising administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, to the subject, thereby treating the MYCC-associated DLBCL in the subject in need thereof. Particular embodiments include the steps of (a) determining MYCC gene copy number, or MYCC gene chromosomal location site, in a DLBCL sample from the subject; and (b) administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, to the subject if MYCC gene copy number in the DLBCL sample is increased relative to that of a MYCC gene copy number reference, or if MYCC gene chromosomal location site in the DLBCL sample is translocated relative to that of a MYCC gene chromosomal location site reference. Certain embodiments include administering YM155, or an analog or derivative thereof, to the subject in combination with a second anti-cancer agent selected from CHOP (cyclophosphamide, doxorubicin, vincristine, and prednisone), an anti-CD20 antibody, bendamustine, and an PARP inhibitor, including combinations thereof, optionally R-CHOP (ritixumab+CHOP). In some embodiments, the anti-CD20 antibody is selected from one or more of rituximab, ofatumumab, ocrelizumab, Iodine-131 tositumomab, obinutuzumab, and ibritumomab. In particular embodiments, the PARP inhibitor is selected from one or more of olaparib, rucaparib, nirparib, talazoparib, talazoparib, veliparib, and pamiparib. In certain embodiments, the DLBCL expresses or overexpresses BCL2, BCL6, or both, and/or wherein the DLBCL comprises an alteration of the BCL2 gene (18q21 locus), an alteration of the BCL-6 gene (3q27 locus), or both. Thus, some embodiments further comprise (c) determining BCL2 and/or BCL6 expression in the DLBCL sample from the subject, and/or determining alterations of the BCL2 gene (18q21 locus) and/or the BCL6 gene (3q27 locus) in the DLBCL sample from the subject. Certain of these and related embodiments comprise administering a second anti-cancer agent comprising a BCL2 inhibitor to the subject if the DLBCL expresses or overexpresses BCL2, and/or if the DLBCL comprises an alteration of the BCL2 gene (18q21 locus). In some embodiments, the BCL2 inhibitor is selected from ABT199 (venetoclax), ABT-263 (navitoclax), ABT-737, GX-15-070 (obatoclax), GDC-0199, BP1002 (antisense), AT-101, and SPC2996. In some embodiments, the YM155 monobromide, or the analog or derivative thereof, and the second anti-cancer agent are administered separately or sequentially. In some embodiments, the YM155 monobromide, or the analog or derivative thereof, and the second anti-cancer agent are administered together at the same time, for example, as part of same or different compositions. Some embodiments include administering to the subject a chemotherapeutic agent excluding (or other than) YM155 monobromide if MYCC gene copy number in the DLBCL sample is not substantially increased relative to that of the MYCC gene copy number reference, or if MYCC gene chromosomal location site in the DLBCL sample is not translocated relative to that of the MYCC gene chromosomal location site reference.

Also included are methods for treating a MYCC/BCL2-associated diffuse large B-cell lymphoma (DLBCL) in a subject in need thereof, comprising administering YM155 monobromide [1-(2-Methoxyethyl)-2-methyl-4,9-dioxo-3-(pyrazin-2-ylmethyl)-4,9-dihydro-1H-naphtho[2,3-d]imidazolium bromide], or an analog or derivative thereof, thereby treating the MYCC/BCL2-associated DLBCL in the subject in need thereof. Some embodiments comprise the steps of (a) determining MYCC gene copy number, or MYCC gene chromosomal location site, in the DLBCL sample from the subject; (b) determining BCL2 expression in the DLBCL sample from the subject, and/or determining alterations of the BCL2 gene (18q21 locus) in the DLBCL sample from the subject; and (c) administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, to the subject if MYCC gene copy number in the DLBCL sample is increased relative to that of a MYCC gene copy number reference, or if MYCC gene chromosomal location site in the DLBCL sample is translocated relative to that of a MYCC gene chromosomal location site reference, and if the DLBCL expresses or overexpresses BCL2, and/or if the DLBCL comprises an alteration of the BCL2 gene (18q21 locus). Particular embodiments include administering YM155, or an analog or derivative thereof, to the subject in combination with a second anti-cancer agent selected from a BCL2 inhibitor, CHOP (cyclophosphamide, doxorubicin, vincristine, and prednisone), an anti-CD20 antibody, bendamustine, and an PARP inhibitor, including combinations thereof, optionally R-CHOP (ritixumab+CHOP). In some embodiments, the BCL2 inhibitor is selected from ABT199 (venetoclax), ABT-263 (navitoclax), ABT-737, GX-15-070 (obatoclax), GDC-0199, BP1002 (antisense), AT-101, and SPC2996. In some embodiments, the anti-CD20 antibody is selected from one or more of rituximab, ofatumumab, ocrelizumab, Iodine-131 tositumomab, obinutuzumab, and ibritumomab. In specific embodiments, the PARP inhibitor is selected from one or more of olaparib, rucaparib, nirparib, talazoparib, talazoparib, veliparib, and pamiparib.

The “MYC gene” or “MYC oncogene” refers to a family of proto-oncogenes that encode transcription factors, examples of which include c-Myc (also MYCC) and N-myc (also MYCN).

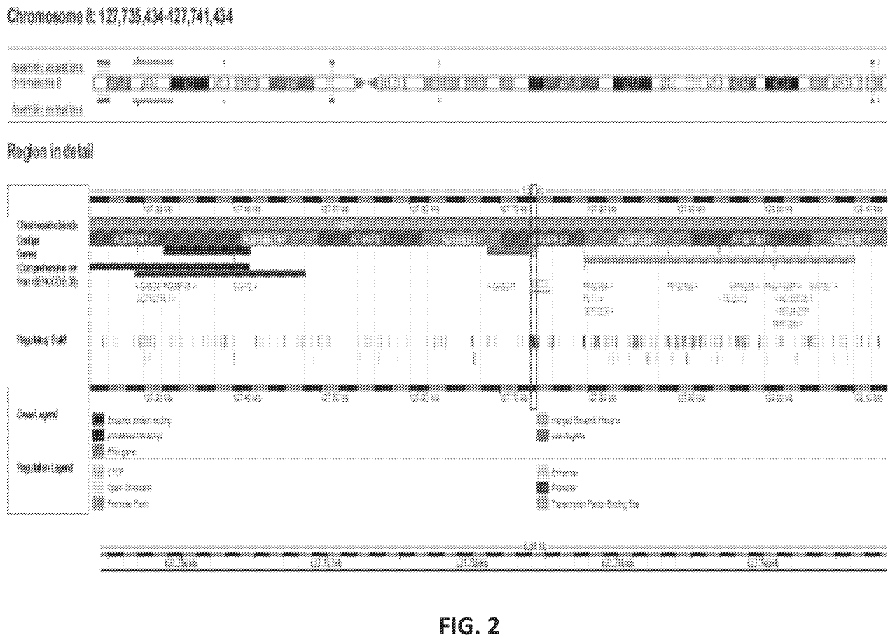

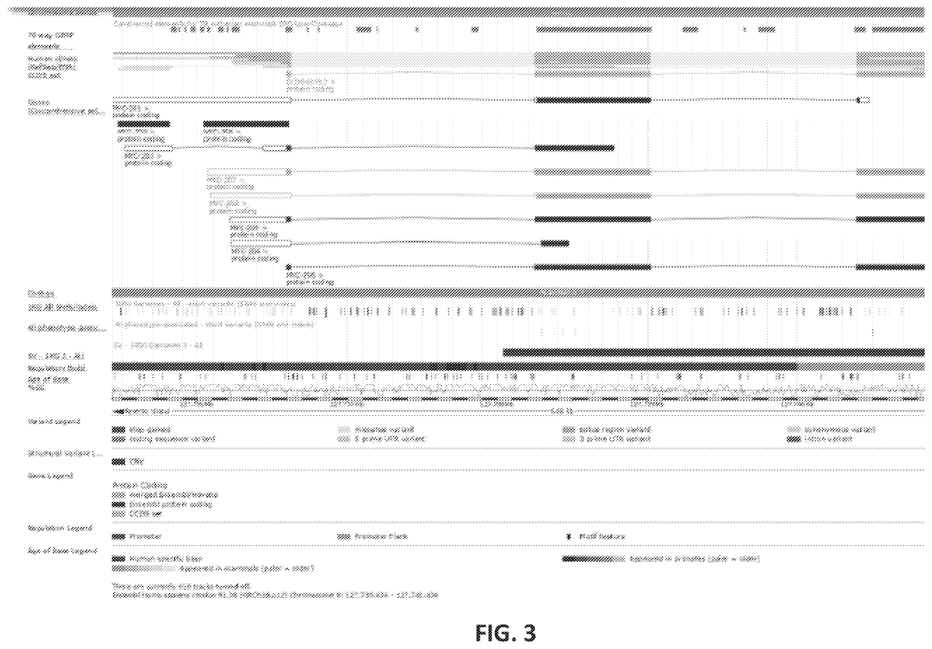

The MYCC gene encodes a nuclear phosphoprotein that plays a role in cell cycle progression, apoptosis, and cellular transformation. The encoded protein forms a heterodimer with the related transcription factor MAX. This complex binds to the E box DNA consensus sequence and regulates the transcription of specific target genes. There is evidence to show that translation initiates both from an upstream, in-frame non-AUG (CUG) and a downstream AUG start site, resulting in the production of two isoforms with distinct N-termini. In the human genome, the MYCC gene is located on chromosome 8:127, 735, 434-127, 741, 434, forward strand (see, e.g., FIG. 2 and FIG. 3; and Gene: MYC ENSG00000136997).

The feature “MYCC-associated cancer”, including a “MYCC-associated DLBCL”, refers to a cancer in which MYCC gene copy number in the cancer is increased relative to that of a MYCC gene copy number reference, and/or a cancer in which MYCC gene chromosomal location site in the cancer is translocated relative to that of a MYCC gene chromosomal location site reference, as described herein. Specific embodiments refer to a DLBCL sample in which the MYCC gene copy number in the DLBCL sample is increased relative to that of a MYCC gene copy number reference, or if MYCC gene chromosomal location site in the DLBCL sample is translocated relative to that of a MYCC gene chromosomal location site reference.

BCL2 (B-Cell Lymphoma 2), encoded in humans by the BCL2 gene, is a member of the BCL2 family of regulator proteins that regulate cell death (apoptosis), by either inhibiting (anti-apoptotic) or inducing (pro-apoptotic) apoptosis.

The feature “BCL2-associated cancer”, including “BLC2-associated DLBCL” or “BCL2-positive DLBCL”, refers to a cancer that expresses, overexpresses, or abnormally expresses BCL2, and/or comprises one or more genetic alterations in the BCL2 gene (18q21 locus). Merely for illustrative purposes, antibodies directed against BCL2 can be used to identify cancers cells that express BCL2, that is, BLC-2-positive DLBCL, for example, by immunohistochemistry (IHC). In healthy tissue, anti-BCL2 antibodies react mainly with B-cells in the mantle zone. In contrast, the number of BCL2-positive cells increase considerably in DLBCL (see, for example, Tsuyama et al., Blood. 130(4):489-500, 2017).

B-cell lymphoma 6 protein is encoded by the BCL6 gene, and has clinical significance in lymphoma. Like BCL2, the presence of BCL6 can be demonstrated in tissue sections using immunohistochemistry. It is exclusively present in the B-cells of both healthy and neoplastic germinal centres. It therefore demonstrates both reactive hyperplasia in lymph nodes and a range of lymphomas derived from follicular B-cells (see, for example, Ye et al., Science. 262 (5134): 747-50, 1993).

“YM155 monobromide” refers to the small molecule [1-(2-Methoxyethyl)-2-methyl-4,9-dioxo-3-(pyrazin-2-ylmethyl)-4,9-dihydro-1H-naphtho[2,3-d] imidazolium bromide], having the molecular formula C₂₀H₁₉N₄O₃.Br, and the CAS Number 781661-94-7, and includes pharmaceutically-acceptable salts and acids thereof. Also included are biologically-active or equivalent analogs and/or derivatives of YM155 monobromide.

As noted above, in some instances, the MYCC gene copy number in the cancer tissue (e.g., DLBCL sample) is increased relative to that of the MYCC gene copy number reference. In particular embodiments, the MYCC gene copy number in the cancer tissue (e.g., DLBCL sample) is increased by a statistically significant amount relative to that of the MYCC gene copy number reference. In some embodiments, the MYCC gene copy number in the cancer tissue (e.g., DLBCL sample) is increased by about or at least about 1.2, 1.3, 1.4, 1.5, 1.6, 1.7, 1.8, 1.9, 2, 2.5, 3, 3.5, 4, 4.5, 5, 6, 7, 8, 9, or 10-fold (or more) relative to that of the MYCC gene copy number reference.

The MYCC gene copy number in the cancer tissue (e.g., DLBCL sample) can be determined by any variety of methods. For example, in some embodiments, the MYCC gene copy number is determined by array comparative genome hybridization (aCGH), single nucleotide polymorphism (SNP) array, copy number variation (CNV) sequencing, or multiplex ligation-dependent probe amplification (MLPA). Certain embodiments thus include the step of determining or detecting copy number of a MYCC gene in a sample of cancer tissue from a subject in need thereof, for example, a DLBCL sample. Also included is the step of comparing the copy number of a MYCC gene in a sample of cancer tissue (e.g., DLBCL sample) relative to that of a MYCC gene copy number reference.

The MYCC gene chromosomal location site in the cancer tissue (e.g., DLBCL sample) can be determined by any variety of methods. For example, in some embodiments, the MYCC gene chromosomal location site in the cancer tissue is determined by in situ hybridization (ISH), fluorescence in situ hybridization (FISH), next generation sequencing (NGS), or comparative genome hybridization (CGH). Certain embodiments thus include the step of determining or detecting the MYCC gene chromosomal location site in a sample of cancer tissue from a subject in need thereof. Also included is the step of comparing the MYCC gene chromosomal location site in the cancer tissue relative to that of a MYCC gene chromosomal site reference.

CGH refers to a molecular cytogenetic method for analyzing copy number variations (CNVs) relative to ploidy level in the DNA of a test sample compared to a reference sample, without the need for culturing cells. This technique allows quick and efficient comparisons between two genomic DNA samples arising from two sources, which are most often closely related, because it is suspected that they contain differences in terms of either gains or losses of either whole chromosomes or subchromosomal regions (a portion of a whole chromosome). The technique was originally developed for the evaluation of the differences between the chromosomal complements of solid tumor and normal tissue (see, e.g., Kallioniemi et al., Science. 258 (5083): 818-821, 1992). The use of DNA microarrays in conjunction with CGH techniques has led to the development of a more specific form of array CGH (aCGH), allowing for a locus-by-locus measure of CNV with increased resolution as low as 100 kilobases (see, e.g., Pinkel, Annu Rev Genom Hum Genet. 6:331-354, 2005). CNV is a prevalent form of critical genetic variation that leads to an abnormal number of copies of large genomic regions in a cell, and high-resolution sequence data can be analyzed by next-generation sequencing (NGS) to identify the same (see, e.g., Zhao et al., BMC Bioinformatics. 14 Suppl 11:S1, 2013). MLPA refers to a variation of the multiplex polymerase chain reaction that permits amplification of multiple targets with only a single primer pair (see, e.g., Schouten et al., Nucleic Acids Res. 30 (12): e57, 2002). In situ hybridization (ISH) and fluorescent in situ hybridization (FISH) refer to a type of hybridization that uses a labeled complementary DNA, RNA or modified nucleic acids strand (i.e., probe) to localize a specific DNA or RNA sequence in a portion or section of tissue (in situ) (see, e.g., Parra & Windle, Nature Genetics. 5:17-21, 1993; and Gall & Pardue, PNAS USA. 63: 378-383, 1969). Thus, in some instances, the methods and kits described herein employ any one or more of the foregoing techniques and/or comprise reagents for performing the same.

Examples of a “reference” (e.g., a MYCC gene copy number “reference”, a MYCC gene chromosomal site “reference”, a BCL2 “reference”) include a value or amount or location obtained from a database, for example, a value or amount of a “wild-type” MYCC gene copy number, a “wild-type” MYCC gene chromosomal location site (see, e.g., FIG. 2 and FIG. 3 for a human MYCC gene chromosomal site reference), or a wild-type BCL2 gene. A “reference” also includes a value or amount or location obtained from a non-cancerous tissue from one or more controls, for example, one or more healthy or non-cancerous control subjects (e.g., a population of healthy or non-cancerous control subjects), or one or more corresponding non-cancerous control tissues from the subject being tested. Typically, a “corresponding” non-cancerous control tissue is obtained from the same type of tissue as the cancer tissue being tested, for example, a non-cancerous B-cell. As with the cancer tissue, the MYCC gene copy number reference and/or the BCL2 reference from a non-cancerous control can be determined by any variety of methods, including, for example, by aCGH, SNP array, CNV sequence, and/or MLPA (supra). Similarly, the MYCC gene chromosomal location site reference and/or BCL2 expression levels/alterations from a non-cancerous control can be determined by any variety of methods, including, for example, ISH, FISH, NGS, and/or CGH (supra).

In some embodiments, the sample of cancer tissue (or non-cancerous control tissue) is a surgical sample, a biopsy sample, a pleural effusion sample, or an ascetic fluid sample from the subject. Particular examples of samples of cancer tissues (or non-cancerous control tissues) include blood, lymph node, and bone marrow samples, particularly those that comprise B-cells. Certain embodiments include the step of obtaining the sample of cancer tissue (or non-cancerous control tissue) from the subject, for example, prior to determining MYCC gene copy levels, MYCC gene chromosomal location site, BCL2 expression levels, and/or BCL2 gene alterations.

In some embodiments, the subject is a human subject.

In some embodiments, as noted above, the cancer or tumor, for example, DLBCL, is a metastatic cancer, that is, a metastatic form of DLBCL. In some embodiments, the DLBCL is selected from DLBCL not otherwise specified (DLBCL-NOS), T-cell/histiocyte-rich B-cell lymphoma, primary or secondary DLBCL of the central nervous system (CNS), primary cutaneous DLBCL (leg type), and Epstein-Barr virus (EBV)-positive DLBCL optionally of the elderly.

In some embodiments, the DLBCL is refractory to at least one prior therapy, including a therapy selected from any one or more of radiation therapy, surgery (optionally radiosurgery), chemotherapy, and immunotherapy. The term “refractory” refers to a tumor or cancer that does not respond to therapy (or treatment). In some instances, the tumor was resistant at the beginning of the prior treatment(s), and in some instances, it became resistant during the prior treatment(s). Particular examples of prior therapies include anthracycline-based therapy, CHOP, anti-CD20 therapy (optionally rituximab), R-CHOP (ritixumab+CHOP), radiation therapy, etoposide (optionally R-EPOCH), and autologous stem cell transplant (ASCT), including combinations thereof.

As noted above, certain embodiments include administering to the subject an anti-cancer agent excluding (or other than) YM155 monobromide if the subject is characterized as non-responsive to YM155 monobromide therapy, for example, if the MYCC gene copy number in the cancer tissue is not substantially increased relative to that of the MYCC gene copy number reference, or if the MYCC gene chromosomal location site in the cancer tissue is not translocated relative to that of the MYCC gene chromosomal location site reference. Exemplary anti-cancer agents (other than YM155 monobromide) for administering to a subject characterized as non-responsive to YM155 monobromide therapy include small molecules such as cytotoxic, chemotherapeutic, and anti-angiogenic agents, for instance, those that have been considered useful in the treatment of various cancers. General classes of anti-cancer agents include, without limitation, alkylating agents, anti-metabolites, anthracyclines, anti-tumor antibiotics, platinums, type I topoisomerase inhibitors, type II topoisomerase inhibitors, vinca alkaloids, and taxanes. Additional examples of anti-cancer agents for administering to a subject characterized as non-responsive to YM155 monobromide therapy include anti-hormonal agents that act to regulate or inhibit hormone action on tumors.

In certain embodiments, the methods described herein are sufficient to result in tumor regression, as indicated by a statistically significant decrease in the amount of viable tumor, for example, at least a 10%, 20%, 30%, 40%, 50% or greater decrease in tumor mass or tumor volume, or by altered (e.g., decreased with statistical significance) scan dimensions. In certain embodiments, the methods described are sufficient to result in stable disease. In certain embodiments, the methods described herein are sufficient to result in clinically relevant reduction in symptoms of a particular disease indication known to the skilled clinician.

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Methods for identifying subjects with one or more of the diseases or conditions described herein are known in the art.

For in vivo use, for instance, for the treatment of human disease or testing, the agents described herein are generally incorporated into one or more therapeutic or pharmaceutical compositions prior to administration.

To prepare a therapeutic or pharmaceutical composition, an effective or desired amount of one or more agents is typically mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations according to the present invention are prepared so that a parenteral dosage unit contains between 0.01 to 10% by weight of the agent of interest prior to dilution.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

Certain embodiments include the use of a diagnostic kit for determining or predicting a therapeutic response (or responsiveness) to YM155 monobromide [1-(2-Methoxyethyl)-2-methyl-4,9-dioxo-3-(pyrazin-2-ylmethyl)-4,9-dihydro-1H-naphtho[2,3-d] imidazolium bromide] therapy in a subject with diffuse large B-cell lymphoma (DLBCL), comprising means for measuring MYCC gene copy number, or MYCC gene chromosomal location site, in a sample of DLBCL tissue from the subject, including cancer tissue and non-cancerous tissue; and a means for determining BCL2 expression in the DLBCL sample of tissue from the subject, and/or means for determining alterations of the BCL2 gene (18q21 locus) in the sample of tissue from the subject.

Also included are patient care kits, comprising: (a) means for measuring MYCC gene copy number, or MYCC gene chromosomal location site, in a diffuse large B-cell lymphoma (DLBCL) sample of tissue from a subject, including cancer tissue and non-cancerous tissue; (b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide]; (b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof; and (c) a second anti-cancer agent selected from CHOP (cyclophosphamide, doxorubicin, vincristine, and prednisone), an anti-CD20 antibody, bendamustine, a PARP inhibitor, and a BCL2 inhibitor, including combinations thereof, optionally R-CHOP (ritixumab+CHOP). In some embodiments, the anti-CD20 antibody is selected from one or more of rituximab, ofatumumab, ocrelizumab, Iodine-131 tositumomab, obinutuzumab, and ibritumomab. In some embodiments, the PARP inhibitor is selected from one or more of olaparib, rucaparib, nirparib, talazoparib, talazoparib, veliparib, and pamiparib. In some embodiments, the BCL2 inhibitor is selected from ABT199 (venetoclax), ABT-263 (navitoclax), ABT-737, GX-15-070 (obatoclax), GDC-0199, BP1002 (antisense), AT-101, and SPC2996. Certain kits further comprise (d) means for determining BCL2 expression in the sample of tissue from the subject, and/or means for determining alterations of the BCL2 gene (18q21 locus) in the sample of tissue from the subject.

Certain embodiments include patient care kits, comprising (a) means for measuring MYCC gene copy number, or MYCC gene chromosomal location site, in a diffuse large B-cell lymphoma (DLBCL) sample of tissue from a subject, including cancer tissue and non-cancerous tissue; (b) means for determining BCL2 expression in the DLBCL sample of tissue from the subject, and/or means for determining alterations of the BCL2 gene (18q21 locus) in the sample of tissue from the subject; and (c) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof. Certain kits further comprise (d) a second anti-cancer agent selected from CHOP (cyclophosphamide, doxorubicin, vincristine, and prednisone), an anti-CD20 antibody, bendamustine, a PARP inhibitor, and a BCL2 inhibitor, including combinations thereof, optionally R-CHOP (ritixumab+CHOP). In some embodiments, the anti-CD20 antibody is selected from one or more of rituximab, ofatumumab, ocrelizumab, Iodine-131 tositumomab, obinutuzumab, and ibritumomab. In some embodiments, the PARP inhibitor is selected from one or more of olaparib, rucaparib, nirparib, talazoparib, talazoparib, veliparib, and pamiparib. In some embodiments, the BCL2 inhibitor is selected from ABT199 (venetoclax), ABT-263 (navitoclax), ABT-737, GX-15-070 (obatoclax), GDC-0199, BP1002 (antisense), AT-101, and SPC2996.

In some embodiments, the means for measuring MYCC gene copy number comprise reagents for performing a diagnostic assay selected from one or more of array comparative genome hybridization (aCGH), single nucleotide polymorphism (SNP) array, copy number variation (CNV) sequencing, and multiplex ligation-dependent probe amplification (MLPA) on a human MYCC gene. In some embodiments, the means for measuring MYCC gene chromosomal location site comprise reagents for performing a diagnostic assay selected from one or more of in situ hybridization (ISH), fluorescence in situ hybridization (FISH), next generation sequencing (NGS), and comparative genome hybridization (CGH) on a human MYCC gene.

In some embodiments, the means for determining BCL2 expression in the DLBCL sample of tissue from the subject, and/or means for determining alterations of the BCL2 gene (18q21 locus) in the sample of tissue from the subject, are selected from one or more of aCGH, SNP array, CNV sequencing, MLPA on a human BCL2 gene, and reagents for performing a diagnostic assay selected from one or more of ISH, FISH, NGS, CGH on a human BCL2 gene, and immunohistochemistry (IHC).

Certain diagnostic or patient care kits include a MYCC gene copy number reference value obtained from a database, or determined from a non-cancerous tissue from a control. Some diagnostic or patient care kits include a MYCC gene chromosomal location site reference obtained from a database, or determined from a non-cancerous tissue from a control. The kits can also include written instructions, for example, on how to determine MYCC gene copy number and/or a MYCC gene chromosomal location site in a sample of cancer tissue from a subject, and/or from a non-cancerous control. Certain diagnostic or patient care kits include a BCL2 expression reference value obtained from a database, or determined from a non-cancerous tissue from a control. Some diagnostic or patient care kits include a BCL2 wild-type sequence or other genetic information obtained from a database, or determined from a non-cancerous tissue from a control. The kits can also include written instructions, for example, on how to determine BCL2 expression levels or gene alterations in a sample of cancer tissue from a subject, and/or from a non-cancerous control.

In some embodiments, a diagnostic or patient care kit contains separate containers, dividers, or compartments for the composition(s) and informational material(s). For example, the composition(s) or reagents can be contained in a bottle, vial, or syringe, and the informational material(s) can be contained in association with the container. In some embodiments, the separate elements of the kit are contained within a single, undivided container. For example, the composition(s) or reagents are contained in a bottle, vial or syringe that has attached thereto the informational material in the form of a label. In some embodiments, the kit includes a plurality (e.g., a pack) of individual containers, each containing one or more compositions, reagents, and/or unit dosage forms of YM155 monobromide. For example, the kit includes a plurality of syringes, ampules, foil packets, or blister packs, each containing a reagent or a single unit dose of YM155 monobromide. The containers of the kits can be air tight, waterproof (e.g., impermeable to changes in moisture or evaporation), and/or light-tight.

The patient care kit optionally includes a device suitable for administration of the agent(s), e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

In certain aspects, the diagnostic or therapeutic response tests or methods described herein are performed at a diagnostic laboratory, and the results are then provided to the subject, or to a physician or other healthcare provider that plays a role in the subject's healthcare and cancer treatment. Particular embodiments thus include methods for providing the results of the responsiveness test to the subject in need thereof, or to the physician or other healthcare provider. These results or data can be in the form of a hard-copy or paper-copy, or an electronic form, such as a computer-readable medium.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill certain changes and modifications may be made thereto without departing from the spirit or scope of the description or appended claims. The following examples are provided by way of illustration only and not by way of limitation. Those of skill in the art will readily recognize a variety of noncritical parameters that could be changed or modified to yield essentially similar results.

### EXAMPLES

### Example 1

### Activity of YM155 in Models of DLBCL

Studies were performed to evaluate the efficacy of YM155 monobromide in combination with other anti-cancer agents for treating MYCC-associated diffuse large B-cell lymphoma (DLBCL). Studies were also performed to evaluate the use of BCL2 as a secondary biomarker to MYCC for identifying DLBCL patients that will respond to YM155-based therapies, including combinations with other anti-cancer agents such as BCL2 inhibitors.

Materials & Methods

Cell Culture. Human diffuse large B-cell lymphoma cells, SU-DHL-10, SU-DHL-4, OCI-LY-7 and SU-DHL-2 cells (purchased from Cell Bank, Shanghai Institutes for Biological Sciences, Chinese Academy of Sciences) were cultured in RPMI 1640 (Hyclone™, SH30809.01B) supplemented with 10% fetal bovine serum (GEMINI, 900-108). Cultures were incubated at 37° C., in 5% CO₂.

Cell Treatment and proliferation assay. SU-DHL-10, OCI-LY-7 and SU-DHL-2 cell were seeded in 96-well plates (Corning-Costar, 3599) at 40000 cells/well in 200 μl culture medium treated with YM155 (50, 25, 12.5, 2.5, 1, 0.5 nM; Apex BIO, A4221) or DMSO (0.1%; Amresco, 67-68-5). After 72 h incubation, cell proliferation was measured by XTT assay (Cell Proliferation Kit II XTT, sigma, 11465015001) according to manufacturer's protocols. Briefly, after YM155 treatment, added to each well 50 μl of the XTT labeling mixture, then incubated the micro-plate for 4 h in a humidified atmosphere. The absorbance of wavelength was determined at 490 nm (OD₄₉₀) and 650 nm (OD₆₅₀) respectively by SpectraMax 190. The IC50 was calculated by the value of OD₄₉₀. OD₆₅₀.

Apoptosis Assay. Apoptosis was detected by Annexin V/PI staining kit (Thermo Fisher) according to the manufacturer's instructions. Briefly, cells were plated at a density of 5×10⁵ cells/mL in RPMI 1640 media with 2% FBS with desired concentrations of YM155. At 24 hours post-treatment the cells were harvested and tested for apoptosis by Annexin V and PI staining. Cell analysis was performed using a FACSAria (BD).

Western blot. SU-DHL-10, OCI-LY-7 and SU-DHL-2 cells were treated with/without YM155 for 24 hours. Cell lysates were prepared in in lysis buffer (Thermo Fisher). Protein concentrations were determined using a Bio-Rad protein assay (Bio-Rad). 20 μg protein was loaded in 4%-12% SDS-PAGE (Invitrogen), and transferred to nitrocellulose. Membranes were blocked in 5% BSA (Sigma) for 1 hour, incubated with the primary antibody overnight at 4° C., washed and incubated with the appropriate horseradish-conjugated secondary antibody (1:5000, Cell Signaling Technology) for 1 hour at room temperature. An enhanced chemiluminescent kit (Beyotime Biotechnology) was used to visualize the signal. The following antibodies were used: c-MYC (Abcam) and GAPDH (Beyotime Biotechnology).

Tumor Xenograft models (CDX models). BALB/C-nu mice (6-8 weeks, SPF Biotechnology Co., Ltd) were inoculated by s.c. injection of tumor cell (SU-DHL-10, SU-DHL-4 or OCI-LY-7) suspension containing Matrigel (Corning)/PBS at a ratio of 1:1. 1×10⁷ cells were inoculated. When tumors reached 150-200 mm³, treatment was initiated. Tumor growth was monitored twice a week. Tumor volume was calculated by applying the following equation: tumor volume=½(length×width²).

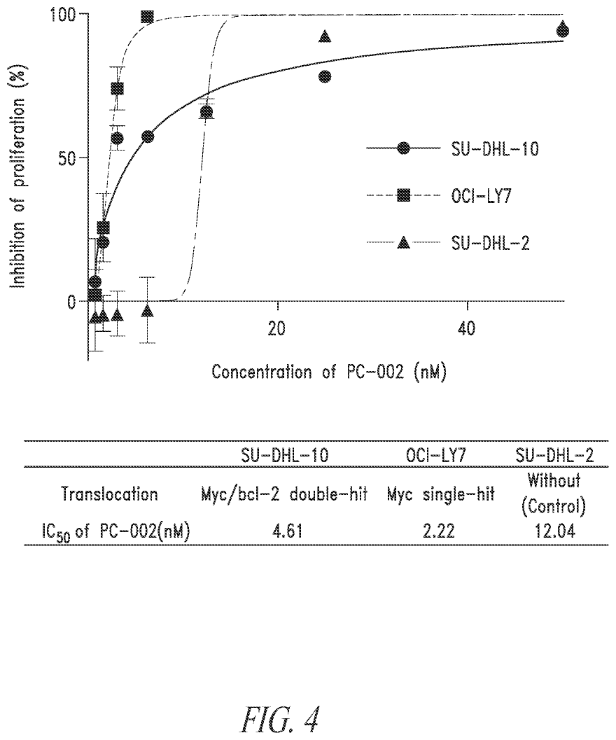

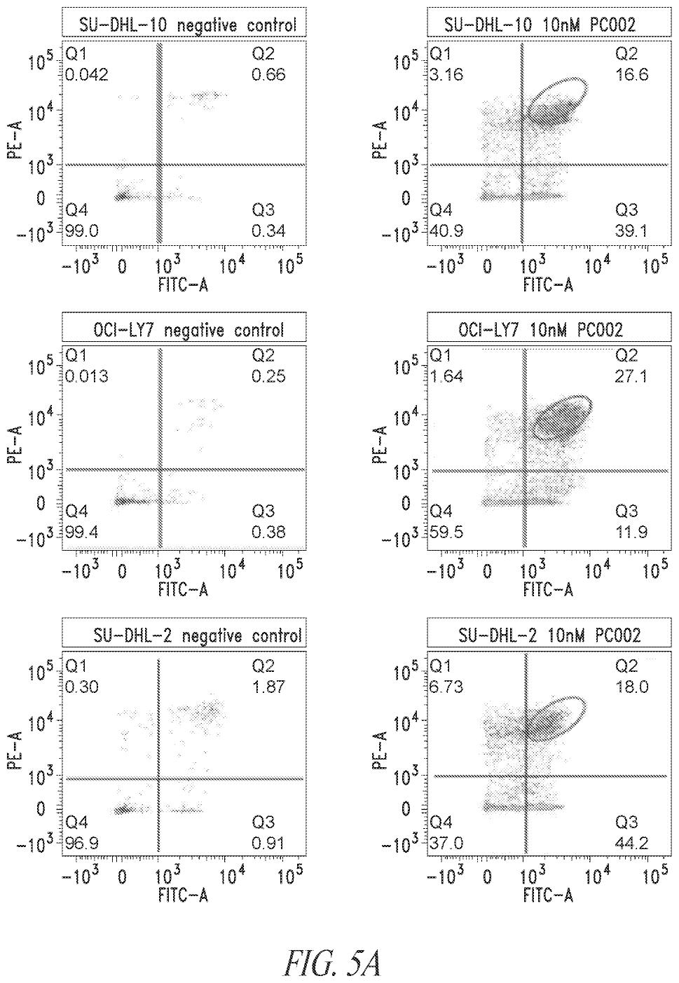

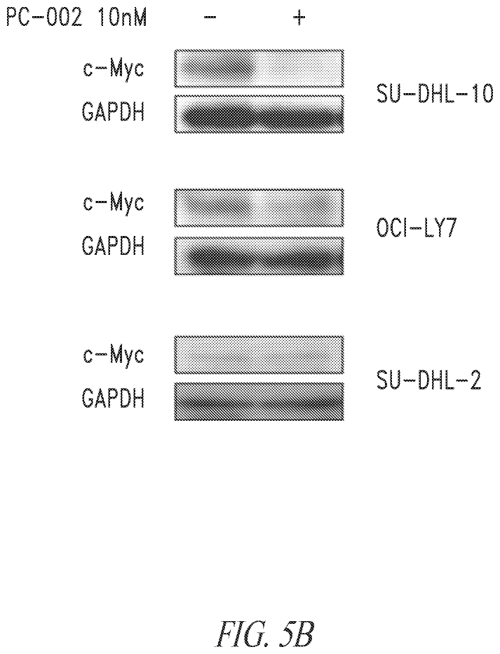

The results are shown in FIGS. 4-10. FIG. 4 shows that DLBCL with a MYCC rearrangement (MYCC-associated DLBCL) is more sensitive to YM155 (PC-002) than DLBCL without the MYCC rearrangement. Similarly, FIG. 5A shows that YM155 (PC-002) induces apoptosis of DLBCL cells with a MYCC rearrangement, and FIG. 5B shows that YM155 (PC-002) lowers the MYCC expression in DLBCL cells with a MYCC rearrangement.

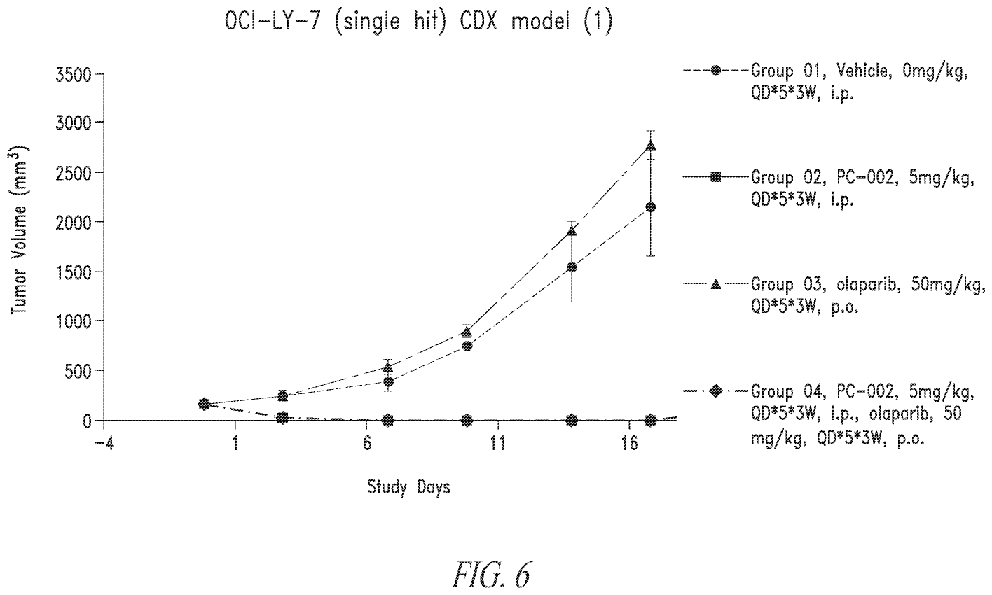

FIG. 6 shows that YM155 (PC-002) causes tumor regression in a cell-line derived xenograft (CDX) mouse model of DLBCL with a MYCC rearrangement, alone and in combination with a PARP inhibitor (olaparib). The PARP inhibitor alone had no significant effect on tumor growth. Thus, combination therapies with YM155 and PARP inhibitors could provide significantly improved utility in the treatment of DLBCL.

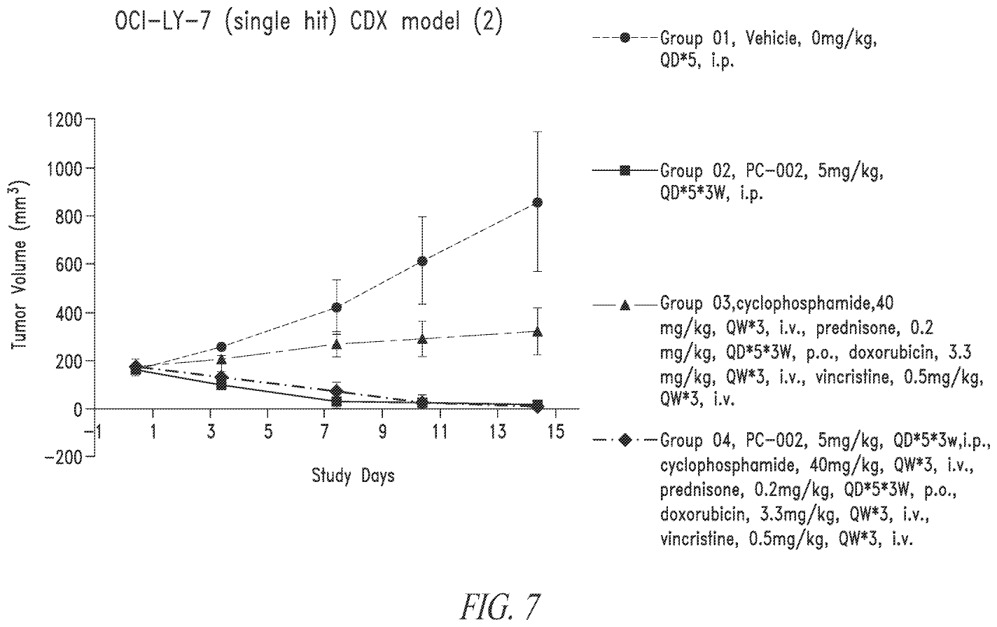

FIG. 7 shows that YM155 (PC-002) causes tumor regression in a CDX mouse model of DLBCL with a MYCC rearrangement, alone and in combination with CHOP, the standard of care for DLBCL. CHOP therapy alone had only a moderate effect on tumor growth. Combination therapies with YM155 and CHOP or R-CHOP (rituximab+CHOP) could provide significantly improved utility in the treatment of DLBCL relative to CHOP or R-CHOP alone.

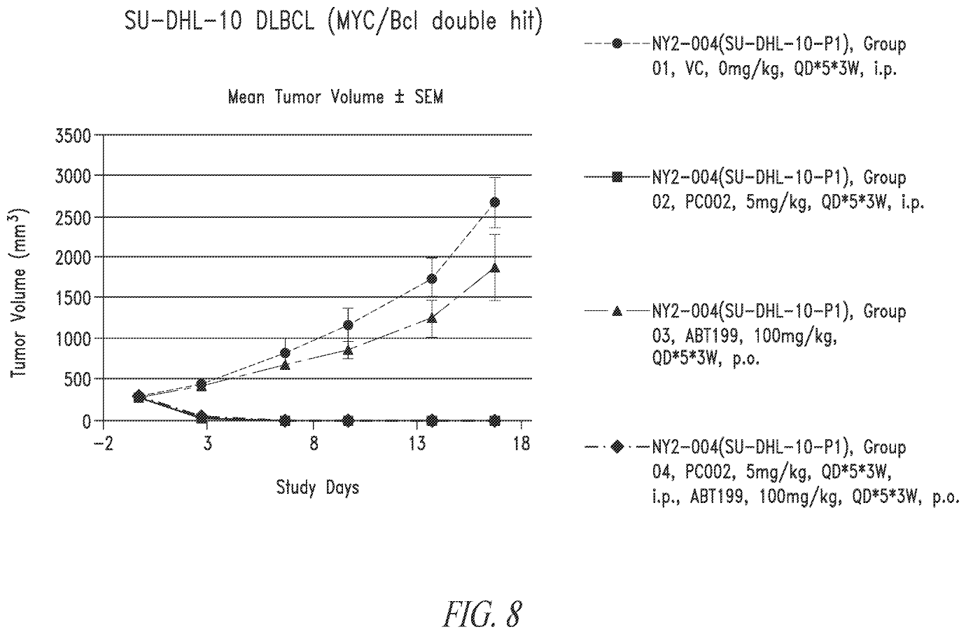

FIG. 8 shows that YM155 (PC-002) causes complete tumor regression in a CDX mouse model of DLBCL with a MYCC rearrangement and BCL2 expression (MYCC/BCL2 associated lymphoma), alone and in combination with a BCL2 inhibitor (ABT199). The BCL2 inhibitor alone had no significant effect on tumor growth. These data show that the combination of MYCC and BCL2 biomarkers could find significant utility in identifying DLBCL that will respond positively to YM155-based therapies, including combination therapies with BCL2 inhibitors and others.

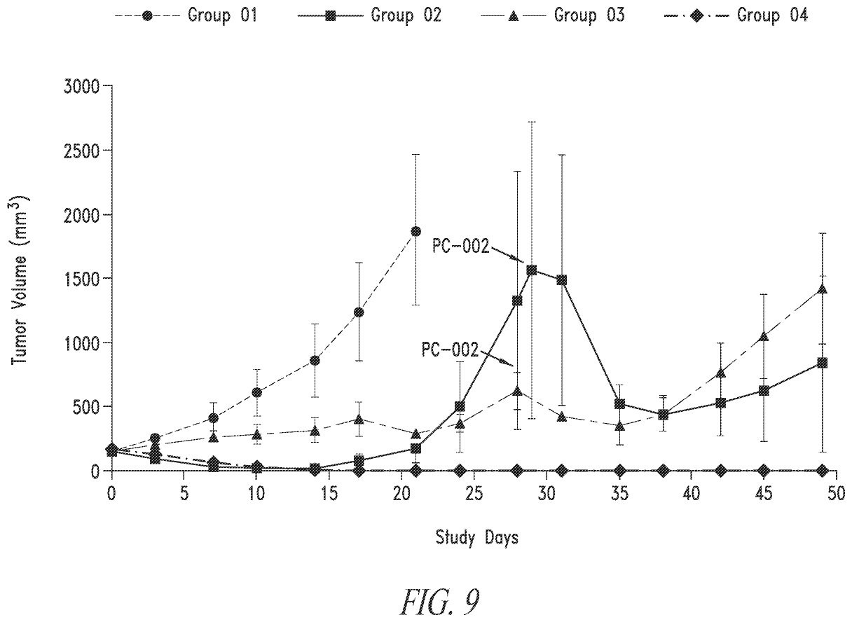

FIG. 9 shows the results of YM155 (PC-002) treatment in a CDX model of DLBCL with a MYCC rearrangement (b/c-nu), alone and in combination with CHOP. Group 1 is vehicle control, Group 2 is YM155 (PC-002) only, Group 3 is CHOP only, and Group 4 is YM155+CHOP. Here, each YM155 and CHOP had a significant effect on reducing tumor volume in the CDX model of DLBCL with a MYCC rearrangement, and the combination of TM155+CHOP had a profound effect on reducing tumor volume, essentially eliminating tumor growth in this DLBCL model.

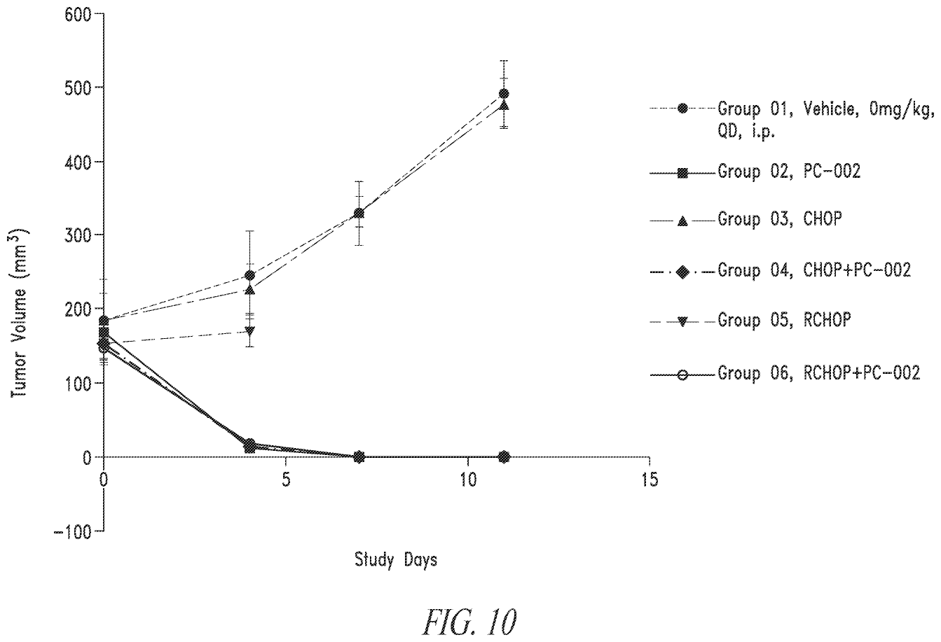

FIG. 10 shows the results of YM155 (PC-002) treatment in a SU-DHL-4 (double hit (MYCC/BCL2), CD20 positive) CDX model (b/c-nu), alone and in combination with CHOP or RCHOP (rituximab+CHOP). Here, neither CHOP nor RCHOP had a significant effect in reducing tumor volume in the CDX model of DLBCL with a MYCC rearrangement and a BCL2 alteration. In contrast, YM155 alone or in combination with CHOP or RCHOP had a profound effect on reducing tumor volume, essentially eliminating tumor growth in this DLBCL model.

1. A method for treating a MYCC-associated diffuse large B-cell lymphoma (DLBCL) in a subject in need thereof, comprising
administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof,
thereby treating the MYCC-associated DLBCL in the subject in need thereof.

2. The method of claim 1, comprising,
(a) determining MYCC gene copy number, or MYCC gene chromosomal location site, in a DLBCL sample from the subject; and
(b) administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, to the subject if MYCC gene copy number in the DLBCL sample is increased relative to that of a MYCC gene copy number reference, or if MYCC gene chromosomal location site in the DLBCL sample is translocated relative to that of a MYCC gene chromosomal location site reference.

3. The method of claim 1 or 2, comprising administering to the subject a chemotherapeutic agent excluding (or other than) YM155 monobromide if MYCC gene copy number in the DLBCL sample is not substantially increased relative to that of the MYCC gene copy number reference, or if MYCC gene chromosomal location site in the DLBCL sample is not translocated relative to that of the MYCC gene chromosomal location site reference.

4. The method of claim 1 or 2, comprising administering YM155, or an analog or derivative thereof, to the subject in combination with a second anti-cancer agent selected from CHOP (cyclophosphamide, doxorubicin, vincristine, and prednisone), an anti-CD20 antibody, bendamustine, and an PARP inhibitor, including combinations thereof, optionally R-CHOP (ritixumab+CHOP).

5. The method of claim 4, wherein the anti-CD20 antibody is selected from one or more of rituximab, ofatumumab, ocrelizumab, Iodine-131 tositumomab, obinutuzumab, and ibritumomab, or wherein the PARP inhibitor is selected from one or more of olaparib, rucaparib, nirparib, talazoparib, talazoparib, veliparib, and pamiparib.

6. The method of any one of claims 1-5, wherein the MYCC gene copy number in the DLBCL sample is increased by about or at least about 1.5, 2, 3, 4, 5, 6, 7, 8, 9, or 10-fold relative to that of the MYCC gene copy number reference.

7. The method of any one of claims 1-6, comprising determining MYCC gene copy number in the DLBCL sample by array comparative genome hybridization (aCGH), single nucleotide polymorphism (SNP) array, copy number variation (CNV) sequencing, or multiplex ligation-dependent probe amplification (MLPA).

8. The method of any one of claims 1-7, comprising determining MYCC gene chromosomal location site in the DLBCL sample by in situ hybridization (ISH), fluorescence in situ hybridization (FISH), next generation sequencing (NGS), or comparative genome hybridization (CGH).

9. The method of any one of claims 1-8, comprising obtaining the MYCC gene copy number reference from a database, or determining the MYCC gene copy number reference from a non-cancerous tissue (optionally B-cell) from a control, optionally by aCGH, SNP array, CNV sequence, or MLPA.

10. The method of any one of claims 1-9, comprising obtaining the MYCC gene chromosomal location site reference from a database, or determining the MYCC gene chromosomal location site reference from a non-cancerous tissue (optionally B-cell) from a control, optionally by ISH, FISH, NGS, or CGH.

11. The method of any one of claims 1-10, comprising obtaining the DLBCL sample from the subject.

12. The method of any one of claims 1-11, wherein the DLBCL sample is a surgical sample, a biopsy sample, a pleural effusion sample, or an ascetic fluid sample obtained from the subject, optionally selected from one or more of blood, lymph node, and bone marrow.

13. The method of any one of claims 1-12, wherein the subject is a human subject.

14. The method of any one of claims 1-13, wherein the DLBCL is selected from DLBCL not otherwise specified (DLBCL-NOS), T-cell/histiocyte-rich B-cell lymphoma, primary or secondary DLBCL of the central nervous system (CNS), primary cutaneous DLBCL (leg type), and Epstein-Barr virus (EBV)-positive DLBCL optionally of the elderly.

15. The method of any one of claims 1-14, wherein the DLBCL is refractory to prior therapy, optionally selected from anthracycline-based therapy, CHOP, anti-CD20 therapy (optionally rituximab), R-CHOP (ritixumab+CHOP), radiation therapy, etoposide (optionally R-EPOCH), and autologous stem cell transplant (ASCT), including combinations thereof.

16. The method of any one of claims 1-13, wherein the DLBCL expresses or overexpresses BCL2, BCL6, or both, and/or wherein the DLBCL comprises an alteration of the BCL2 gene (18q21 locus), an alteration of the BCL-6 gene (3q27 locus), or both.

17. The method of claim 16, further comprising,
(c) determining BCL2 and/or BCL6 expression in the DLBCL sample from the subject, and/or determining alterations of the BCL2 gene (18q21 locus) and/or the BCL6 gene (3q27 locus) in the DLBCL sample from the subject.

18. The method of claim 16 or 17, further comprising administering a second anti-cancer agent comprising a BCL2 inhibitor to the subject if the DLBCL expresses or overexpresses BCL2, and/or if the DLBCL comprises an alteration of the BCL2 gene (18q21 locus), optionally wherein the BCL2 inhibitor is selected from ABT199 (venetoclax), ABT-263 (navitoclax), ABT-737, GX-15-070 (obatoclax), GDC-0199, BP1002 (antisense), AT-101, and SPC2996.

19. The method of any one of claims 4-18, wherein the YM155 monobromide, or the analog or derivative thereof, and the second anti-cancer agent are administered separately or sequentially.

20. The method of any one of claims 4-18, wherein the YM155 monobromide, or the analog or derivative thereof, and the second anti-cancer agent are administered together at the same time.

21. A method for treating a MYCC/BCL2-associated diffuse large B-cell lymphoma (DLBCL) in a subject in need thereof, comprising
administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof,
thereby treating the MYCC/BCL2-associated DLBCL in the subject in need thereof.

22. The method of claim 21, comprising
(a) determining MYCC gene copy number, or MYCC gene chromosomal location site, in the DLBCL sample from the subject;
(b) determining BCL2 expression in the DLBCL sample from the subject, and/or determining alterations of the BCL2 gene (18q21 locus) in the DLBCL sample from the subject; and
(c) administering YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof, to the subject if MYCC gene copy number in the DLBCL sample is increased relative to that of a MYCC gene copy number reference, or if MYCC gene chromosomal location site in the DLBCL sample is translocated relative to that of a MYCC gene chromosomal location site reference, and if the DLBCL expresses or overexpresses BCL2, and/or if the DLBCL comprises an alteration of the BCL2 gene (18q21 locus).

23. The method of claim 21 or 22, comprising administering YM155, or an analog or derivative thereof, to the subject in combination with a second anti-cancer agent selected from a BCL2 inhibitor, CHOP (cyclophosphamide, doxorubicin, vincristine, and prednisone), an anti-CD20 antibody, bendamustine, and an PARP inhibitor, including combinations thereof, optionally R-CHOP (ritixumab+CHOP).

24. The method of claim 23, wherein the BCL2 inhibitor is selected from ABT199 (venetoclax), ABT-263 (navitoclax), ABT-737, GX-15-070 (obatoclax), GDC-0199, BP1002 (antisense), AT-101, and SPC2996.

25. The method of claim 23, wherein the anti-CD20 antibody is selected from one or more of rituximab, ofatumumab, ocrelizumab, Iodine-131 tositumomab, obinutuzumab, and ibritumomab, or wherein the PARP inhibitor is selected from one or more of olaparib, rucaparib, nirparib, talazoparib, talazoparib, veliparib, and pamiparib.

26. The method of any one of claims 21-25, wherein the MYCC gene copy number in the DLBCL sample is increased by about or at least about 1.5, 2, 3, 4, 5, 6, 7, 8, 9, or 10-fold relative to that of the MYCC gene copy number reference.

27. The method of any one of claims 21-26, comprising determining MYCC gene copy number in the DLBCL sample by array comparative genome hybridization (aCGH), single nucleotide polymorphism (SNP) array, copy number variation (CNV) sequencing, or multiplex ligation-dependent probe amplification (MLPA).

28. The method of any one of claims 21-27, comprising determining MYCC gene chromosomal location site in the DLBCL sample by in situ hybridization (ISH), fluorescence in situ hybridization (FISH), next generation sequencing (NGS), or comparative genome hybridization (CGH).

29. The method of any one of claims 21-28, comprising obtaining the MYCC gene copy number reference from a database, or determining the MYCC gene copy number reference from a non-cancerous tissue (optionally B-cell) from a control, optionally by aCGH, SNP array, CNV sequence, or MLPA.

30. The method of any one of claims 21-29, comprising obtaining the MYCC gene chromosomal location site reference from a database, or determining the MYCC gene chromosomal location site reference from a non-cancerous tissue (optionally B-cell) from a control, optionally by ISH, FISH, NGS, or CGH.

31. The method of any one of claims 21-30, comprising obtaining the DLBCL sample from the subject.

32. The method of any one of claims 21-31, wherein the DLBCL sample is a surgical sample, a biopsy sample, a pleural effusion sample, or an ascetic fluid sample obtained from the subject, optionally selected from one or more of blood, lymph node, and bone marrow.

33. The method of any one of claims 21-32, wherein the subject is a human subject.

34. The method of any one of claims 21-33, wherein the DLBCL is selected from DLBCL not otherwise specified (DLBCL-NOS), T-cell/histiocyte-rich B-cell lymphoma, primary or secondary DLBCL of the central nervous system (CNS), primary cutaneous DLBCL (leg type), and Epstein-Barr virus (EBV)-positive DLBCL optionally of the elderly.

35. The method of any one of claims 21-34, wherein the DLBCL is refractory to prior therapy, optionally selected from anthracycline-based therapy, CHOP, anti-CD20 therapy (optionally rituximab), R-CHOP (ritixumab+CHOP), radiation therapy, etoposide (optionally R-EPOCH), and autologous stem cell transplant (ASCT), including combinations thereof.

36. A patient care kit, comprising:
(a) means for measuring MYCC gene copy number, or MYCC gene chromosomal location site, in a diffuse large B-cell lymphoma (DLBCL) sample of tissue from a subject, including cancer tissue and non-cancerous tissue;
(b) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof; and
(c) a second anti-cancer agent selected from CHOP (cyclophosphamide, doxorubicin, vincristine, and prednisone), an anti-CD20 antibody, bendamustine, a PARP inhibitor, and a BCL2 inhibitor, including combinations thereof, optionally R-CHOP (ritixumab+CHOP).

37. The patient care kit of claim 36, wherein the anti-CD20 antibody is selected from one or more of rituximab, ofatumumab, ocrelizumab, Iodine-131 tositumomab, obinutuzumab, and ibritumomab, or wherein the PARP inhibitor is selected from one or more of olaparib, rucaparib, nirparib, talazoparib, talazoparib, veliparib, and pamiparib, or wherein the BCL2 inhibitor is selected from ABT199 (venetoclax), ABT-263 (navitoclax), ABT-737, GX-15-070 (obatoclax), GDC-0199, BP1002 (antisense), AT-101, and SPC2996.

38. The patient care kit of claim 36 or 37, wherein the means for measuring MYCC gene copy number comprise reagents for performing a diagnostic assay selected from one or more of array comparative genome hybridization (aCGH), single nucleotide polymorphism (SNP) array, copy number variation (CNV) sequencing, and multiplex ligation-dependent probe amplification (MLPA) on a human MYCC gene.

39. The patient care kit of any one of claims 36-38, wherein the means for measuring MYCC gene chromosomal location site comprise reagents for performing a diagnostic assay selected from one or more of in situ hybridization (ISH), fluorescence in situ hybridization (FISH), next generation sequencing (NGS), and comparative genome hybridization (CGH) on a human MYCC gene.

40. The patient care kit of any one of claims 36-39, comprising a MYCC gene copy number reference value obtained from a database, or determined from a non-cancerous tissue from a control.

41. The patient care kit of any one of claims 36-40, comprising a MYCC gene chromosomal location site reference obtained from a database, or determined from a non-cancerous tissue from a control.

42. The patient care kit of any one of claims 36-41, comprising (d) means for determining BCL2 expression in the sample of tissue from the subject, and/or means for determining alterations of the BCL2 gene (18q21 locus) in the sample of tissue from the subject.

43. A patient care kit, comprising:
(a) means for measuring MYCC gene copy number, or MYCC gene chromosomal location site, in a diffuse large B-cell lymphoma (DLBCL) sample of tissue from a subject, including cancer tissue and non-cancerous tissue;
(b) means for determining BCL2 expression in the DLBCL sample of tissue from the subject, and/or means for determining alterations of the BCL2 gene (18q21 locus) in the sample of tissue from the subject; and
(c) YM155 monobromide [1-(2-Methoxyethyl)-2-methyl-4,9-dioxo-3-(pyrazin-2-ylmethyl)-4,9-dihydro-1H-naphtho[2,3-d] imidazolium bromide], or an analog or derivative thereof.

44. The patient care kit of claim 43, comprising (d) a second anti-cancer agent selected from CHOP (cyclophosphamide, doxorubicin, vincristine, and prednisone), an anti-CD20 antibody, bendamustine, a PARP inhibitor, and a BCL2 inhibitor, including combinations thereof, optionally R-CHOP (ritixumab+CHOP).

45. The patient care kit of claim 43 or 44, wherein the anti-CD20 antibody is selected from one or more of rituximab, ofatumumab, ocrelizumab, Iodine-131 tositumomab, obinutuzumab, and ibritumomab, or wherein the PARP inhibitor is selected from one or more of olaparib, rucaparib, nirparib, talazoparib, talazoparib, veliparib, and pamiparib, or wherein the BCL2 inhibitor is selected from ABT199 (venetoclax), ABT-263 (navitoclax), ABT-737, GX-15-070 (obatoclax), GDC-0199, BP1002 (antisense), AT-101, and SPC2996.

46. The patient care kit of any one of claims 43-45, wherein the means for measuring MYCC gene copy number comprise reagents for performing a diagnostic assay selected from one or more of array comparative genome hybridization (aCGH), single nucleotide polymorphism (SNP) array, copy number variation (CNV) sequencing, and multiplex ligation-dependent probe amplification (MLPA) on a human MYCC gene.

47. The patient care kit of any one of claims 43-46, wherein the means for measuring MYCC gene chromosomal location site comprise reagents for performing a diagnostic assay selected from one or more of in situ hybridization (ISH), fluorescence in situ hybridization (FISH), next generation sequencing (NGS), and comparative genome hybridization (CGH) on a human MYCC gene

48. The patient care kit of any one of claims 43-47, comprising a MYCC gene copy number reference value obtained from a database, or determined from a non-cancerous tissue from a control

49. The patient care kit of any one of claims 43-48, comprising a MYCC gene chromosomal location site reference obtained from a database, or determined from a non-cancerous tissue from a control.
